(19) United States
(12) Patent Application Publication
(10) Pub. No.: US 2015/0320575 A1
(43) Pub. Date: Nov. 12, 2015

(54) INTUITIVE PROSTHETIC INTERFACE

(71) Applicant: The Regents of the University of California, Oakland, CA (US)

(72) Inventors: Sanjay Joshi, Walnut Creek, CA (US); Kenneth Lyons, Davis, CA (US)

(73) Assignee: THE REGENTS OF THE UNIVERSITY OF CALIFORNIA, Oakland, CA (US)

(21) Appl. No.: 14/705,872

(22) Filed: May 6, 2015

Related U.S. Application Data
(60) Provisional application No. 61/989,673, filed on May 7, 2014.

(51) Int. Cl. A61F 2/72; A61F 2/60; A61F 2/54

(52) U.S. Cl. CPC A61F 2/72; A61F 2/54; A61F 2/60; A61F 2002/704

(57) ABSTRACT

A prosthetic device is controlled based on measured electrical signals corresponding to muscle activity in a lower-extremity anatomical analog to a dysfunctional or absent portion of an upper extremity of an individual. In particular, an activity monitoring device measures electrical signals corresponding to muscle activity in the lower-extremity anatomical analog. Then, a communication device communicates information specifying the electrical signals to a pattern-recognition device. The pattern-recognition device identifies a natural mode of movement of the lower-extremity anatomical analog based on the information. For example, the natural mode of movement may be identified using a pattern-recognition technique. Next, the pattern-recognition device provides a control signal to a prosthetic device associated with the dysfunctional or absent portion of the upper extremity, where the control signal specifies a natural mode of movement of the prosthetic device that is an analog to the natural mode of movement of the lower-extremity anatomical analog.

### CROSS-REFERENCE TO RELATED APPLICATION

This application claims priority under 35 U.S.C. §119(e) to U.S. Provisional Application Ser. No. 61/989,673, entitled “Intuitive Prosthetic Interface,” by Sanjay Joshi and Kenneth Lyons, Attorney Docket Number UC14-774-1PSP, filed on May 7, 2014, the contents of which are herein incorporated by reference.

### BACKGROUND

1. Field

The described embodiments relate to techniques for controlling a prosthetic device. In particular, the described embodiments relate to techniques for controlling a prosthetic device for a missing or dysfunctional extremity based on muscle activity in an anatomical analog extremity.

2. Related Art

Prosthetic devices (such as an electromechanically controlled arm or leg) offer patients with missing or dysfunctional limbs the opportunity to restore their mobility and functional capability. Consequently, prosthetic devices can have a dramatic impact on these individuals' lives and quality of life.

However, the increased functionality of prosthetic devices often comes with a cost. For persons with an amputation distal to the elbow, electrical activity sensors may be placed on muscles on the remaining limb to control an arm prosthesis. However, for persons with an amputation proximal to the elbow, remaining muscles are typically far more limited and cannot be used for intuitive prosthetic control. In particular, many prosthetic devices have complicated user interfaces that are difficult to use for high-level amputees. For example, in order for high-level amputees to control many arm prostheses, patients typically need to learn a complex set of commands. Moreover, some arm-prostheses systems require nerve surgery and significant recovery time to enable different body parts, such as pectoral muscles, to be used for control.

Consequently, many patients become frustrated by the long learning times and/or the invasive procedures associated with prosthesis devices, and often give up before they can obtain the benefits of the prosthetic devices.

### SUMMARY

The described embodiments relate to a system. This system includes an activity monitoring device that measures electrical signals corresponding to muscle activity in a first extremity anatomical analog to a dysfunctional or absent portion of a second extremity of an individual. Moreover, the system includes a communication device with: a first antenna, and a first interface circuit that, in conjunction with the first antenna, wirelessly communicates information specifying the electrical signals. Furthermore, the system includes a pattern-recognition device (such as a signal processing device or a machine-learning device) with: a second antenna, and a second interface circuit that, in conjunction with the second antenna, receives the information specifying the electrical signals. Additionally, the pattern-recognition device includes a processor that performs the operations of: identifying a natural mode of movement of the first extremity anatomical analog based on the received information, where the identifying involves a pattern-recognition technique; and provides a control signal to a prosthetic device associated with the dysfunctional or absent portion of the second extremity, where the control signal specifies a natural mode of movement of the prosthetic device that is an analog to the natural mode of movement of the first extremity anatomical analog.

Note that the first extremity anatomical analog and the dysfunctional or absent portion of the second extremity may be on a common side relative to a midline of an individual (such as a right side of the individual or a left side of the individual). Moreover, the first extremity anatomical analog and the dysfunctional or absent portion of the second extremity may be: an N^{th} toe and an N^{th} finger (where N is an integer); a foot and a hand; a distal portion of a leg and a distal portion of an arm; and/or a leg and an arm. Thus, the first extremity may be a lower extremity and the second extremity may be an upper extremity (or vice versa).

Furthermore, the electrical signals may be associated with nerve signals to muscles in the first extremity anatomical analog. For example, the activity monitoring device may include electromyography sensors. In some embodiments, the electromyography sensors are associated with: a tibialis anterior muscle, a peroneus longus muscle, an extensor hallucis longus muscle, an extensor digitorum longus muscle, a flexor digitorum longus muscle, and/or a gastrocnemius lateralis muscle.

Alternatively, the electrical signals may be associated with movement of the first extremity anatomical analog. For example, the activity monitoring device may include: a strain gauge, an accelerometer, and/or a positioning sensor.

In some embodiments, the natural mode of movement of the first extremity anatomical analog is identified based on predefined natural modes of movement of the first extremity anatomical analog.

Note that the natural mode of movement of the first extremity anatomical analog and/or the natural mode of movement of the prosthetic device may include: flexion, extension, abduction, adduction, elevation, depression, rotation, pronation, supination, inversion, eversion, hand open, hand close, and/or chuck grip.

Another embodiment provides a computer-program product for use with the system. This computer-program product includes instructions for at least some of the operations performed by the system.

Another embodiment provides a method for providing the control signal, which may be performed by the system. During operation, the activity monitoring device measures the electrical signals corresponding to the muscle activity in the first extremity anatomical analog to the dysfunctional or absent portion of the second extremity of the individual. Then, the communication device communicates the information specifying the electrical signals to the pattern-recognition device. Moreover, the pattern-recognition device identifies the natural mode of movement of the first extremity anatomical analog based on the information, where the identifying involves a pattern-recognition technique. Next, the pattern-recognition device provides the control signal to the prosthetic device associated with the dysfunctional or absent portion of the second extremity, where the control signal specifies the natural mode of movement of the prosthetic device that is the analog to the natural mode of movement of the first extremity anatomical analog.

The preceding summary is provided as an overview of some exemplary embodiments and to provide a basic understanding of aspects of the subject matter described herein. Accordingly, the above-described features are merely examples and should not be construed as narrowing the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating components in a system wirelessly communicating in accordance with an embodiment of the present disclosure.

FIG. 2 is a flow diagram illustrating a method for providing a control signal in the system of FIG. 1 in accordance with an embodiment of the present disclosure.

FIG. 3 is a drawing illustrating communication among the components in the system of FIG. 1 in accordance with an embodiment of the present disclosure.

FIG. 4 is a drawing illustrating control of an upper-extremity prosthetic device using a lower extremity anatomical analog in accordance with an embodiment of the present disclosure.

FIG. 5 is a drawing illustrating identifying one or more natural modes of movement of a first extremity anatomical analog in FIG. 1 in accordance with an embodiment of the present disclosure.

FIG. 6 is a block diagram illustrating an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A prosthetic device is controlled by a system based on measured electrical signals corresponding to muscle activity in a lower-extremity anatomical analog to a dysfunctional or absent portion of an upper extremity of an individual. In particular, an activity monitoring device measures electrical signals corresponding to muscle activity in the lower-extremity anatomical analog. Then, a communication device communicates information specifying the electrical signals to a pattern-recognition device. The pattern-recognition device identifies a natural mode of movement of the lower-extremity anatomical analog based on the information. For example, the natural mode of movement may be identified using a pattern-recognition technique. Next, the pattern-recognition device provides a control signal to a prosthetic device associated with the dysfunctional or absent portion of the upper extremity, where the control signal specifies a natural mode of movement of the prosthetic device that is an analog to or that corresponds to the natural mode of movement of the lower-extremity anatomical analog.

By using the natural mode of movement of the lower-extremity anatomical analog to control the natural mode of movement of the prosthetic device, the system may provide an intuitive interface. This intuitive interface may be easy for the individual to learn and use. Consequently, the intuitive interface may reduce user frustration or, alternatively, increase user satisfaction, and thus may make it more likely that the individual will use the prosthetic device. In addition, by using the natural mode of movement of the lower-extremity anatomical analog, the intuitive interface may reduce or eliminate the need for invasive procedures, such as surgery, which may reduce the cost and the complexity of the system.

In the discussion that follows, components of the system (such as the communication device and the pattern-recognition device) include radios that communicate the information (e.g., via packets) in accordance with a communication protocol, such as an Institute of Electrical and Electronics Engineers (IEEE) 802.11 standard (which is sometimes referred to as ‘Wi-Fi®,’ from the Wi-Fi Alliance of Austin, Tex.), Bluetooth® (from the Bluetooth Special Interest Group of Kirkland, Wash.), and/or another type of wireless interface. In the discussion that follows, Bluetooth is used as an illustrative example. However, a wide variety of communication protocols may be used.

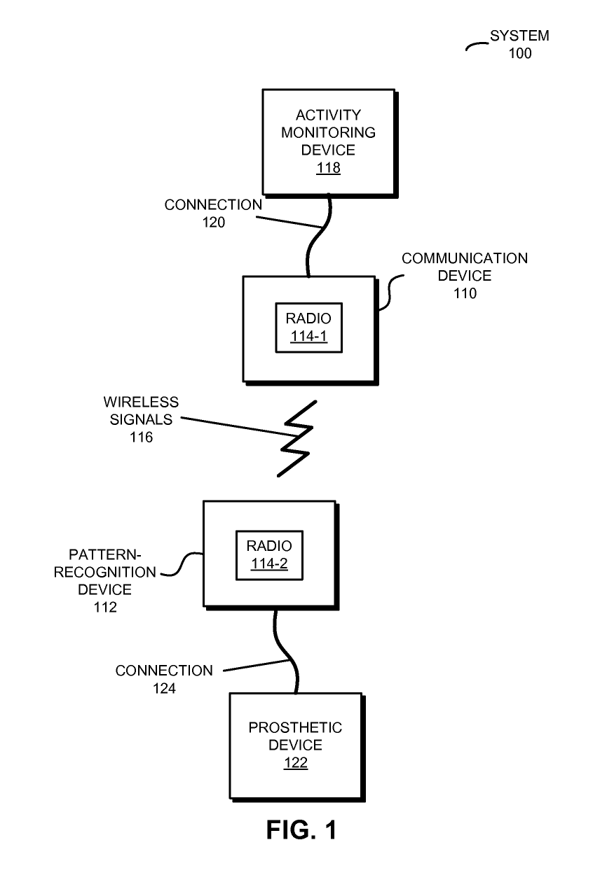

Communication among components in system 100 is shown in FIG. 1, which presents a block diagram illustrating communication device 110 wirelessly communicating with pattern-recognition device 112 (which may be a portable electronic device, e.g., a cellular telephone, which may include or implement a signal-processing technique and/or a machine-learning technique). In particular, these electronic devices may wirelessly communicate while: transmitting advertising frames on wireless channels, detecting one another by scanning wireless channels, establishing connections (for example, by transmitting association requests), and/or transmitting and receiving packets (which may include the association requests and/or additional information as payloads).

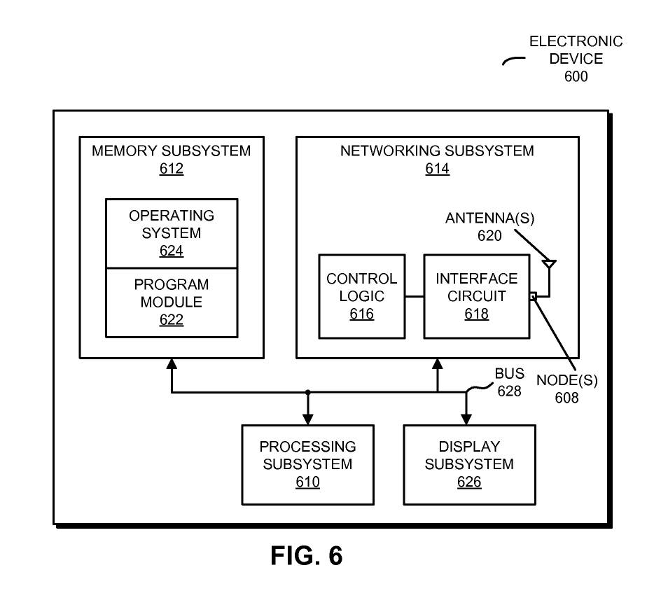

As described further below with reference to FIG. 6, communication device 110 and pattern-recognition device 112 may include subsystems, such as a networking subsystem, a memory subsystem and a processor subsystem. In addition, communication device 110 and pattern-recognition device 112 may include radios 114 in the networking subsystems. More generally, communication device 110 and pattern-recognition device 112 can include (or can be included within) any electronic devices with the networking subsystems that enable communication device 110 and pattern-recognition device 112 to wirelessly communicate with each other. This wireless communication can comprise transmitting advertisements on wireless channels to enable communication device 110 and pattern-recognition device 112 to make initial contact or detect each other, followed by exchanging subsequent data/management frames (such as association requests and responses) to establish a connection, configure security options (e.g., an encryption technique or a secure connection), transmit and receive packets or frames via the connection, etc.

Moreover, as can be seen in FIG. 1, wireless signals 116 (represented by jagged lines) are transmitted by radios 114 in communication device 110 and pattern-recognition device 112. For example, radio 114-1 in communication device 110 may transmit information (such as packets) using wireless signals. These wireless signals are received by a radio 114-2 in pattern-recognition device 112). This may allow communication device 110 to communicate information to pattern-recognition device 112. In the described embodiments, processing a packet or frame in communication device 110 and pattern-recognition device 112 includes: receiving wireless signals 116 with the packet or frame; decoding/extracting the packet or frame from the received wireless signals to acquire the packet or frame; and processing the packet or frame to determine the information contained in the packet or frame.

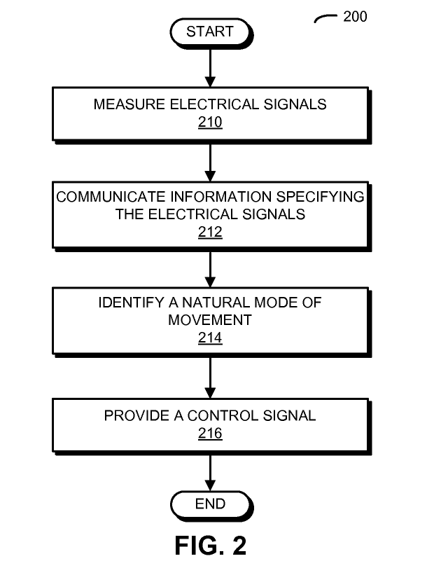

As described further below with reference to FIG. 2, during operation of system 100, activity monitoring device 118, which is electrically coupled to communication device 110 by connection 120 (such as wires or a cable), may monitor or measure or electrical signals corresponding to muscle activity in a first extremity anatomical analog (such as at least a portion of an individual's lower extremity or leg) to a dysfunctional or absent portion of a second extremity of the individual (such as a corresponding portion of the individual's upper extremity or arm). For example, activity monitoring device 118 may include electromyography sensors (such as surface electromyography sensors or sEMG sensors) that measure electrical signals that are associated with nerve signals to muscles in the individual's lower leg. The measurements may include amplification and/or analog-to-digital conversion. Note that sEMG sensors may be included in a compression structure (such as a sock, a sleeve or a shoe) that maintains the positions of the sEMG sensors and/or the contact between the sEMG sensors and the individual. Alternatively, the sEMG sensors may be mechanically coupled to the individual using an adhesive. In addition, note that the sEMG sensors may be electrically coupled to the individual using a conductive gel. (However, in other embodiments, the sEMG sensors do not use a conductive gel. Indeed, in some embodiment the sEMG sensors may not be in physical contact with the individual's skin, which may allow the electrical signals to be measured through clothing. Furthermore, in some embodiments the EMG sensors are secured to the individual's skin more permanently or are implanted into the individual's muscle.) The measured electrical signals may be provided or communicated from activity monitoring device 118 to communication device 110, which may wireless communicate the information specifying the electrical signals to pattern-recognition device 112.

After receiving the information (such as after receiving packets with the information), pattern-recognition device 112 may analyze the information to identify a natural mode of movement of the individual's lower leg using a pattern-recognition technique. For example, pattern-recognition device 112 may extract features from the information and perform a classification operation on the extracted features to recognize specific gestures of the individual's lower leg. In some embodiments, pattern-recognition device 112 identifies the natural mode of movement based on predefined natural modes of movement of the individual's lower leg. Thus, the pattern-recognition technique may include a supervised-learning technique, such as a regression technique, LASSO, linear discriminant analysis, a support vector machine and/or a neural network. However, in other embodiments, the pattern-recognition technique may include an unsupervised-learning technique (i.e., the predefined natural modes of movement may not be used), such as a clustering technique, principal component analysis and, more generally, blind signal separation.

Then, pattern-recognition device 112 may provide a control signal (or a command or instruction) to a prosthetic device 122 (such as a prosthetic or robotic forearm and/or hand) that is electrically coupled to pattern-recognition device 112 by connection 124 (such as wires or a cable). Note that prosthetic device 122 may be associated with the dysfunctional or absent portion of the individual's arm. Moreover, the control signal may specify a natural mode of movement of prosthetic device 122 that is an analog to the natural mode of movement of the individual's leg. For example, the individual may flex or extend their foot (such as dorsiflexion or plantar flexion) to flex or extend a prosthetic hand (such as dorsiflexion or palmarflexion). Alternatively, the individual may rotate their foot to accomplish the same or a similar motion of prosthetic device 122, or may curl or extend their toes to curl or extend artificial fingers in prosthetic device 122. Thus, the individual may mimic the desired motion of prosthetic device 122 using their lower leg, and pattern-recognition device 112 may map or translate this information to a control signal corresponding desired motion of prosthetic device 122 (e.g., using a look-up table). More generally, the first extremity anatomical analog and the dysfunctional or absent portion of the second extremity may be: an N^{th} toe and an N^{th} finger (where N is an integer); a foot and a hand; a distal portion of a leg and a distal portion of an arm; and/or a leg and an arm. In addition, note that the natural mode of movement of the first extremity anatomical analog and/or the natural mode of movement of the prosthetic device may include motions about symmetry axes of the extremities such as: flexion, extension, abduction, adduction, elevation, depression, rotation, pronation, supination, inversion, eversion, hand open, hand close, and/or chuck grip.

In order to maximize the correspondence (or anatomical analog) of the natural modes of the individual's lower leg and prosthetic device 122, the individual's lower leg and the dysfunctional or absent portion of the individual's arm may be on a common side relative to a midline of the individual (such as a right side of the individual or a left side of the individual). Thus, a prosthetic device on a lim may be controlled by a single (anatomical analog) lim. However, in some embodiment, the first extremity anatomical analog is on an opposite side of the body (relative to the midline) from the second extremity.

In these ways, system 100 may provide a simple, intuitive user interface for closed-loop control (i.e., a control or a feedback technique) of prosthetic device 122 by the individual.

While the preceding example illustrated system 100 with at least a portion of the individual's leg being used to control prosthetic device 122 that is associated with at least a portion of the individual's arm, in other embodiments the extremities are reversed, so that at least a portion of the individual's arm is used to control prosthetic device 122 that is associated with at least a portion of the individual's leg. Thus, the first extremity may be a lower extremity and the second extremity may be an upper extremity or vice versa.

Moreover, while the preceding example used sEMG sensors and associated electrical signals to illustrate system 100 and the control technique, a wide variety of sensors may be used, such as another type of EMG sensor (such as an intramuscular EMG sensor) and/or a different physical phenomenon. For example, the electrical signals may be associated with movement or displacement of the first extremity anatomical analog. In particular, activity monitoring device 118 may include: a strain gauge, an accelerometer, and/or a positioning sensor (such as a sensor in a local positioning system). Therefore, activity monitoring device 118 may directly detect motion of the individual's lower leg or foot.

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames. In particular, in some embodiments sensors in prosthetic device 122 (such as a thermal sensor or a pressure sensor) may provide sensory feedback (such as thermal or tactile feedback) to the individual by wirelessly communicating sensory information to the individual's leg, which is then presented or provided to the individual's lower leg via transducers in activity monitoring device 118 (such as a heater or pressure transducer). In some embodiments, connections 120 and/or 124 are replaced with wireless connections or links.

While pattern-recognition device 112 is shown after the wireless communication in FIG. 1, in other embodiments pattern-recognition device 112 is prior to the wireless communication. For example, pattern-recognition device 112 may be included in a smart shoe device that identifies a natural mode of movement before radio 114-1, which may wirelessly communicate control signals.

FIG. 2 presents embodiments of a flow diagram illustrating method 200 for providing a control signal that may be performed by one or more electronic devices, such as pattern-recognition device 112 (FIG. 1), and more generally, system 100 (FIG. 1). During operation, an activity monitoring device measures electrical signals (operation 210) corresponding to muscle activity in a first extremity anatomical analog to a dysfunctional or absent portion of a second extremity of an individual. Then, a communication device communicates information specifying the electrical signals (operation 212) to a pattern-recognition device. Moreover, the pattern-recognition device identifies a natural mode of movement (operation 214) of the first extremity anatomical analog based on the information, where the identifying involves a pattern-recognition technique. Next, the pattern-recognition device provides a control signal (operation 216) to a prosthetic device associated with the dysfunctional or absent portion of the second extremity, where the control signal specifies a natural mode of movement of the prosthetic device that is an analog to or that corresponds to the natural mode of movement of the first extremity anatomical analog.

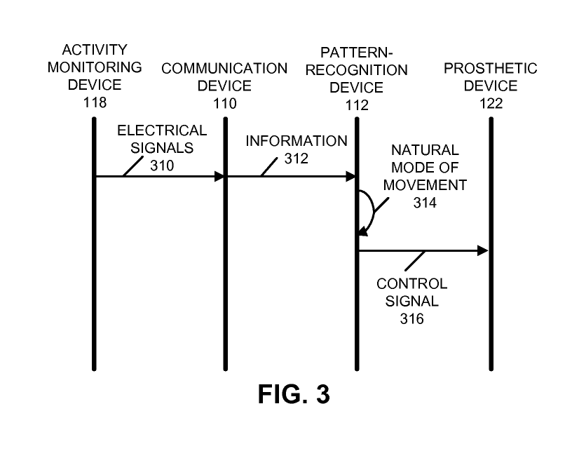

Embodiments of the control technique are further illustrated in FIG. 3, which presents a drawing illustrating communication among components in system 100 (FIG. 1). In particular, during the control technique activity monitoring device 118 may measure electrical signals 310 corresponding to muscle activity in a first extremity anatomical analog to a dysfunctional or absent portion of a second extremity of an individual. These electrical signals 310 may be provided to communication device 110.

Then, communication device 110 may communicate information 312 specifying electrical signals 310 to pattern-recognition device 112. After receiving information 312, pattern-recognition device 112 may identify a natural mode of movement 314 of the first extremity anatomical analog based on information 312.

Next, pattern-recognition device 112 provides a control signal 316 to prosthetic device 122 associated with the dysfunctional or absent portion of the second extremity, where the control signal specifies a natural mode of movement of prosthetic device 122 that is an analog to or that corresponds to the natural mode of movement of the first extremity anatomical analog. In response, prosthetic device 122 performs the specified natural mode of movement.

In this way, the system (e.g., software executed by the pattern-recognition device) may facilitate closed-loop control of the prosthetic device. In particular, the system may improve the ease of use or the intuitive nature of controlling the prosthetic device. For example, the system may improve the accuracy of the outputs (the control signal) based on the inputs to system (such as the identified natural mode of movement of the first extremity anatomical analog). This may reduce user frustration and the learning time needed to learn how to control the prosthetic device and, thus, may improve the user experience when using the prosthetic device.

In some embodiments of method 200 (FIG. 2), there may be additional or fewer operations. Moreover, the order of the operations may be changed, and/or two or more operations may be combined into a single operation.

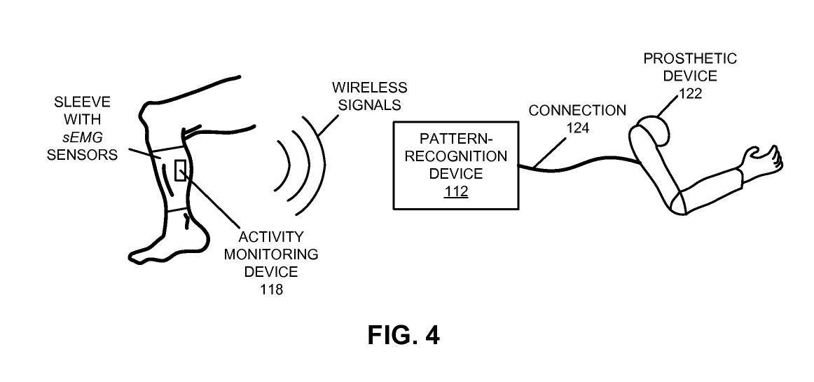

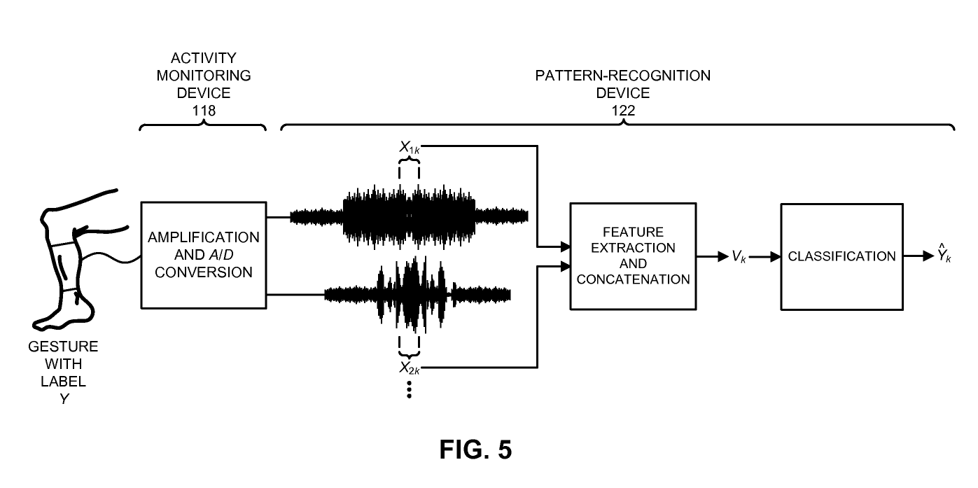

FIG. 4 presents a drawing illustrating control of an upper-extremity prosthetic device using a lower extremity anatomical analog. In particular, sEMG sensors on an individual's lower leg measure electrical signals associated with muscle activity of the lower leg that are used to control motion of the upper-extremity prosthetic device. As shown in FIG. 5, which presents a drawing illustrating identifying one or more natural modes of movement of a first extremity anatomical analog (such as the lower extremity anatomical analog), the one or more natural modes of movement may be identified in the measured electrical signals using a pattern-recognition technique.

In an exemplary embodiment, the sEMG sensors are associated with: a tibialis anterior muscle, a peroneus longus muscle, an extensor hallucis longus muscle, an extensor digitorum longus muscle, a flexor digitorum longus muscle, and/or a gastrocnemius lateralis muscle. While the musculature of the lower leg is somewhat different than the forearm, by activating one or more of these muscles the individual may be able to intuitively control the upper-extremity prosthetic device with little or no training using leg or foot gestures. For example, classification of leg gestures using the control technique with sEMG sensors on the leg may exceed 90%, which is comparable to the accuracy of classification of the analogous arm gestures with sEMG sensors on the arm.

We now describe embodiments of an electronic device (or a component) in system 100 (FIG. 1), such as communication device 110 or pattern-recognition device 112. FIG. 6 presents a block diagram illustrating an electronic device 600, such as one of electronic devices in FIG. 1. This electronic device includes processing subsystem 610, memory subsystem 612, and networking subsystem 614. Processing subsystem 610 includes one or more devices configured to perform computational operations. For example, processing subsystem 610 can include one or more microprocessors, application-specific integrated circuits (ASICs), microcontrollers, programmable-logic devices, and/or one or more digital signal processors (DSPs).

Memory subsystem 612 includes one or more devices for storing data and/or instructions for processing subsystem 610 and networking subsystem 614. For example, memory subsystem 612 can include dynamic random access memory (DRAM), static random access memory (SRAM), and/or other types of memory. In some embodiments, instructions for processing subsystem 610 in memory subsystem 612 include: one or more program modules or sets of instructions (such as program module 622 or operating system 624), which may be executed by processing subsystem 610. Note that the one or more computer programs may constitute a computer-program mechanism. Moreover, instructions in the various modules in memory subsystem 612 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 610.

In addition, memory subsystem 612 can include mechanisms for controlling access to the memory. In some embodiments, memory subsystem 612 includes a memory hierarchy that comprises one or more caches coupled to a memory in electronic device 600. In some of these embodiments, one or more of the caches is located in processing subsystem 610.

In some embodiments, memory subsystem 612 is coupled to one or more high-capacity mass-storage devices (not shown). For example, memory subsystem 612 can be coupled to a magnetic or optical drive, a solid-state drive, or another type of mass-storage device. In these embodiments, memory subsystem 612 can be used by electronic device 600 as fast-access storage for often-used data, while the mass-storage device is used to store less frequently used data.

Networking subsystem 614 includes one or more devices configured to couple to and communicate on a wired and/or wireless network (i.e., to perform network operations), including: control logic 616, an interface circuit 618 and one or more antennas 620. (While FIG. 6 includes one or more antennas 620, in some embodiments electronic device 600 includes one or more nodes 608, e.g., a pad, which can be coupled to one or more antennas 620. Thus, electronic device 600 may or may not include one or more antennas 620.) For example, networking subsystem 614 can include a Bluetooth networking system, a cellular networking system (e.g., a 3G/4G network such as UMTS, LTE, etc.), a universal serial bus (USB) networking system, a networking system based on the standards described in IEEE 802.11 (e.g., a Wi-Fi networking system), and/or another networking system.

Networking subsystem 614 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ between the electronic devices does not yet exist. Therefore, electronic device 600 may use the mechanisms in networking subsystem 614 for performing simple wireless communication between the electronic devices, e.g., transmitting advertising or beacon frames and/or scanning for advertising frames transmitted by other electronic devices as described previously.

Within electronic device 600, processing subsystem 610, memory subsystem 612, and networking subsystem 614 are coupled together using bus 628. Bus 628 may include an electrical, optical, and/or electro-optical connection that the subsystems can use to communicate commands and data among one another. Although only one bus 628 is shown for clarity, different embodiments can include a different number or configuration of electrical, optical, and/or electro-optical connections among the subsystems.

In some embodiments, electronic device 600 includes a display subsystem 626 for displaying information on a display, which may include a display driver and the display, such as a liquid-crystal display, a multi-touch touchscreen, etc.

Electronic device 600 can be (or can be included in) any electronic device with at least one network interface. For example, electronic device 600 can be (or can be included in): a desktop computer, a laptop computer, a subnotebook/netbook, a server, a tablet computer, a smartphone, a cellular telephone, a smart watch, a consumer-electronic device, a portable computing device, and/or another electronic device.

Although specific components are used to describe electronic device 600, in alternative embodiments, different components and/or subsystems may be present in electronic device 600. For example, electronic device 600 may include one or more additional processing subsystems, memory subsystems, networking subsystems, and/or display subsystems. Additionally, one or more of the subsystems may not be present in electronic device 600. Moreover, in some embodiments, electronic device 600 may include one or more additional subsystems that are not shown in FIG. 6. Also, although separate subsystems are shown in FIG. 6, in some embodiments, some or all of a given subsystem or component can be integrated into one or more of the other subsystems or component(s) in electronic device 600. For example, in some embodiments program module 622 is included in operating system 624.

Moreover, the circuits and components in electronic device 600 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 614, such as a radio. Moreover, the integrated circuit may include hardware and/or software mechanisms that are used for transmitting wireless signals from electronic device 600 and receiving signals at electronic device 600 from other electronic devices. Aside from the mechanisms herein described, radios are generally known in the art and hence are not described in detail. In general, networking subsystem 614 and/or the integrated circuit can include any number of radios. Note that the radios in multiple-radio embodiments function in a similar way to the described single-radio embodiments.

In some embodiments, networking subsystem 614 and/or the integrated circuit include a configuration mechanism (such as one or more hardware and/or software mechanisms) that configures the radio(s) to transmit and/or receive on a given communication channel (e.g., a given carrier frequency). For example, in some embodiments, the configuration mechanism can be used to switch the radio from monitoring and/or transmitting on a given communication channel to monitoring and/or transmitting on a different communication channel. (Note that ‘monitoring’ as used herein comprises receiving signals from other electronic devices and possibly performing one or more processing operations on the received signals, e.g., determining if the received signal comprises an advertising frame, etc.)

While a communication protocol compatible with Bluetooth was used as an illustrative example, the described embodiments of the control technique may be used in a variety of network interfaces. Furthermore, while some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the control technique may be implemented using program module 622, operating system 624 and/or in firmware in interface circuit 618. Alternatively or additionally, at least some of the operations in the control technique may be implemented in a physical layer, such as hardware in interface circuit 618.

Moreover, while the preceding embodiments illustrated the use of the control technique with a prosthetic device associated with a dysfunctional or absent portion of an extremity of an individual, in other embodiments the control technique is used for remote control of a drone or robot (i.e., human-guided robotics), such as an anthropomorphic robot. In these embodiments, the individual may have all of their limbs and all of their limbs may be functional. Instead, the control technique may be used to control one or more robotic arms at the same or a remote location (such as a robot that is not attached to or in contract with the individual's body). For example, an astronaut may use one or more legs to control a robot while they are busy using their hands for another task.

Furthermore, in some embodiments the control technique is used to implement physical therapy. For example, a stroke victim may be shown a video showing a desired gesture or motion of their right arm. By moving their right leg, the system may assist the individual in moving their right arm. This assisted control of the individual's right arm may facilitate brain plasticity or retrain the neural pathways that allow, over time, the individual to intuitively relearn how to use or control their right arm on their own (i.e., without using the system or with less assistance provided via the system).

In the preceding description, we refer to ‘some embodiments.’ Note that ‘some embodiments’ describes a subset of all of the possible embodiments, but does not always specify the same subset of embodiments. Moreover, note that the numerical values provided are intended as illustrations of the control technique. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A system, comprising:
an activity monitoring device that, during operation of the system, measures electrical signals corresponding to muscle activity in a first extremity anatomical analog to a dysfunctional or absent portion of a second extremity of an individual;
a communication device, electrically coupled to the activation monitoring device, wherein the communication device includes:
a first antenna; and
a first interface circuit, electrically coupled to the first antenna, which, during operation of the system, wirelessly communicates information specifying the electrical signals;
a pattern-recognition device that includes:
a second antenna;
a second interface circuit, electrically coupled to the second antenna, which, during operation of the system, receives the information specifying the electrical signals; and
a processor that, during operation of the system, performs the operations of:
identifying a natural mode of movement of the first extremity anatomical analog based on the received information, wherein the identifying involves a pattern-recognition technique; and
providing a control signal to a prosthetic device associated with the dysfunctional or absent portion of the second extremity, wherein the control signal specifies a natural mode of movement of the prosthetic device that is an analog to the natural mode of movement of the first extremity anatomical analog.

2. The system of claim 1, wherein the first extremity anatomical analog and the dysfunctional or absent portion of the second extremity are on a common side relative to a midline of an individual.

3. The system of claim 1, wherein the first extremity anatomical analog and the dysfunctional or absent portion of the second extremity are one of: an N^{th} toe and an N^{th} finger; a foot and a hand; a distal portion of a leg and a distal portion of an arm; and a leg and an arm.

4. The system of claim 1, wherein the electrical signals are associated with nerve signals to muscles in the first extremity anatomical analog.

5. The system of claim 1, wherein the activity monitoring device includes electromyography sensors.

6. The system of claim 1, wherein the electromyography sensors are associated with one or more of: a tibialis anterior muscle, a peroneus longus muscle, an extensor hallucis longus muscle, an extensor digitorum longus muscle, a flexor digitorum longus muscle, and a gastrocnemius lateralis muscle.

7. The system of claim 1, wherein the electrical signals are associated with movement of the first extremity anatomical analog.

8. The system of claim 1, wherein the activity monitoring device includes one of: a strain gauge, an accelerometer, and a positioning sensor.

9. The system of claim 1, wherein the natural mode of movement of the first extremity anatomical analog is identified based on predefined natural modes of movement of the first extremity anatomical analog.

10. The system of claim 1, wherein the natural mode of movement of the first extremity anatomical analog and the natural mode of movement of the prosthetic device include one of: flexion, extension, abduction, adduction, elevation, depression, rotation, pronation, supination, inversion, eversion, hand open, hand close, and chuck grip.

11. A computer-program product for use in conjunction with a system, the computer-program product comprising a non-transitory computer-readable storage medium and a computer-program mechanism embedded therein to provide a control signal to a prosthetic device, the computer-program mechanism including:
instructions for receiving information specifying electrical signals that correspond to muscle activity in a first extremity anatomical analog to a dysfunctional or absent portion of a second extremity of an individual;
instructions for identifying a natural mode of movement of the first extremity anatomical analog based on the received information, wherein the identifying involves a pattern-recognition technique; and
instructions providing a control signal to a prosthetic device associated with the dysfunctional or absent portion of the second extremity, wherein the control signal specifies a natural mode of movement of the prosthetic device that is an analog to the natural mode of movement of the first extremity anatomical analog.

12. The computer-program product of claim 11, wherein the first extremity anatomical analog and the dysfunctional or absent portion of the second extremity are on a common side relative to a midline of an individual.

13. The computer-program product of claim 11, wherein the first extremity anatomical analog and the dysfunctional or absent portion of the second extremity are one of: an N^{th} toe and an N^{th} finger; a foot and a hand; a distal portion of a leg and a distal portion of an arm; and a leg and an arm.

14. The computer-program product of claim 11, wherein the electrical signals are associated with nerve signals to muscles in the first extremity anatomical analog.

15. The computer-program product of claim 14, wherein the nerve signals are associated with one or more of: a tibialis anterior muscle, a peroneus longus muscle, an extensor hallucis longus muscle, an extensor digitorum longus muscle, a flexor digitorum longus muscle, and a gastrocnemius lateralis muscle.

16. The computer-program product of claim 11, wherein the electrical signals are associated with movement of the first extremity anatomical analog.

17. The computer-program product of claim 11, wherein the electrical signals are associated with one of: a strain gauge, an accelerometer, a positioning sensor, and an electromyography sensor.

18. The computer-program product of claim 11, wherein the natural mode of movement of the first extremity anatomical analog is identified based on predefined natural modes of movement of the first extremity anatomical analog.

19. The computer-program product of claim 11, wherein the natural mode of movement of the first extremity anatomical analog and the natural mode of movement of the prosthetic device include one of: flexion, extension, abduction, adduction, elevation, depression, rotation, pronation, supination, inversion, eversion, hand open, hand close, and chuck grip.

20. A method for providing a control signal, wherein the method comprises:
using an activity monitoring device, measuring electrical signals corresponding to muscle activity in a first extremity anatomical analog to a dysfunctional or absent portion of a second extremity of an individual;
communicating information specifying the electrical signals to a pattern-recognition device;
using the pattern-recognition device, identifying a natural mode of movement of the first extremity anatomical analog based on the information, wherein the identifying involves a pattern-recognition technique; and
providing the control signal to a prosthetic device associated with the dysfunctional or absent portion of the second extremity, wherein the control signal specifies a natural mode of movement of the prosthetic device that is an analog to the natural mode of movement of the first extremity anatomical analog.
